(19) United States
(12) Patent Application Publication
(10) Pub. No.: US 2024/0021315 A1
(43) Pub. Date: Jan. 18, 2024

(54) IN VITRO METHOD FOR PREDICTING THE RISK OF DEVELOPING A BREAST LATE EFFECT AFTER RADIOTHERAPY

(71) Applicants: INSTITUT RÉGIONAL DU CANCER DE MONTPELLIER, Montpellier (FR); UNIVERSITE DE MONTPELLIER, Montpellier (FR)

(72) Inventors: David Azria, Fontanes (FR); Sophie Gourgou, Jacou (FR)

(73) Assignees: INSTITUT RÉGIONAL DU CANCER DE MONTPELLIER, Montpellier (FR); UNIVERSITE DE MONTPELLIER, Montpellier (FR)

(21) Appl. No.: 16/327,974

(22) PCT Filed: Aug. 31, 2017
(86) PCT No.: PCT/EP2017/071887

§ 371 Date: Feb. 25, 2019

(30) Foreign Application Priority Data
Aug. 31, 2016 (EP) 16306097.3

(51) Int. Cl. G16H 50/30; G16H 20/40; G01N 33/68

(52) U.S. Cl. CPC G16H 50/30; G16H 20/40; G01N 33/6893; G01N 2800/40; G01N 2800/50; G01N 2800/60

(57) ABSTRACT

The present invention is drawn to a new diagnosis method and a calculator for predicting the risk of developing a breast late effect (BLE), which is defined as atrophic skin, telangiectasia, induration (fibrosis), necrosis or ulceration, in a subject after radiotherapy (RT), by using Radiation Induced late effect using T-Lymphocyte Apoptosis (RILA) and clinical parameters. The invention is also drawn to diagnosis kits for the implementation of the method and a nomogram.

### CROSS-REFERENCE TO RELATED APPLICATIONS

This application is a U.S. National Stage Application pursuant to 35 U.S.C. § 371 of international Patent Application PCT/EP2017/071887, filed on Aug. 31, 2017, and published as WO 2018/041960 on Mar. 8, 2018, which claims priority to European Patent. Application 16306097.3, filed on Aug. 31, 2016, all of which are incorporated herein by reference in their entireties for all purposes.

### FIELD OF THE INVENTION

The present invention is drawn to a new diagnosis method and a calculator for predicting the risk of developing a breast late effect (BLE), which is defined as atrophic skin, telangiectasia, induration (fibrosis), necrosis or ulceration, in a subject after radiotherapy (RT), by using Radiation induced late effect using T-Lymphocyte Apoptosis (RILA) and clinical parameters. The invention is also drawn to diagnosis kits for the implementation of the method and a nomogram.

### BACKGROUND OF THE INVENTION

Severe but also moderate toxicities after curative-intent RT, such as a poor cosmetic outcome following breast cancer can have a negative impact on quality of life and a marked effect on subsequent psychological outcome (Al-Ghazal, Fallowfield et al. 1999). A number of factors are known to increase the risk of radiation toxicity including intrinsic radiosensitivity (Azria, Betz et al. 2012). While toxicity risks for populations of patients are known, the determination of an individual's normal tissue radiosensitivity is seldom possible before treatment. Therefore, current practice standards commonly prescribe radiation dose according to clinical scenarios from standard recommendations, without regard to the genotype or phenotype of the individual being irradiated.

In that context, Azria et al. (Azria, Riou et al. 2015) showed that radiosensitivity assay based on flow cytometric assessment of RILA, can significantly predict differences in breast fibrosis between individuals and can be used as a rapid screening for potential hyper-reactive patients to RT. Negative predictive value was found in case of high RILA value and less grade ≥2 breast fibrosis (Ozsahin, Crompton et al. 2005). In addition, all severe breast fibrosis (grade ≥2) were observed in patients with low values of RILA. Further, these results were confirmed with multicentric trial showing a negative predictive value of 91% in case of high RILA value and less grade breast fibrosis (Azria, Riou et al. 2015). Nevertheless, the prediction of this radiosensitivity assay based on RILA exhibit some limitation in term of sensitivity and reliability. Taken alone, RILA is not capable of high prediction in terms of sensitivity and specificity.

Therefore, there is an important need to develop a method of diagnostic predicting the risk of developing BLE after RT having an enhanced sensitivity and reliability.

The publication of Azria et al. 2015 assessed the role of RILA as an independent predictor of breast fibrosis after adjuvant breast radiotherapy in a prospective multicenter trial. But in multivariate analysis, no correlation was found between acute effects and RILA or other clinically relevant parameters. In particular, tobacco smoking parameter was not significant in multivariate regression analysis for fibrosis and relapse as disclosed in this prior art.

The invention now provides a new diagnosis method for predicting the probability of developing a breast late effect (BLE), wherein RILA is combined with clinical parameters, and in particular with tobacco smoking habits and adjuvant hormonotherapy, with an improved global evaluation of the risk of developing a BLE for a patient.

As further disclosed in the illustrative examples, a patient with a MLA (radiation-induced CD8 T-lymphocyte apoptosis) of 20% without any other combined clinical parameter is a patient considered without any BLE or relapse risk at 3 years (risk BLE around 2-3%), whereas with the new in vitro diagnosis method according to the invention, combining RILA and clinical parameters in particular tobacco smoking habit and adjuvant hormonotherapy, the risk of developing a BLE is majored at 16%. And for a patient with a RILA of 12% (risk BLE around 5%) without any other combined clinical parameter will have a majored BLE risk of 8% when combined with tobacco smoking clinical parameter and even a majored BLE risk of 22% when combined with tobacco smoking and adjuvant hormonotherapy.

These data showed the interest and efficiency of the combined approach according to the invention to optimize the global evaluation of the BLE risk for a patient.

### SUMMARY OF THE INVENTION

The present invention is related to an in vitro method for diagnosing the risk of developing breast late effects (BLE) after radiotherapy in a subject comprising the steps of:
a. Determining the values of at Least one biochemical marker from a biologic sample of said subject;
b. Determining the level of at least two clinical parameters;
c. Combining said data through a multivariate Cox function to obtain an end value to determine the risk (probability) of developing BLE;
   wherein a multivariate Cox regression from said multivariate Cox function is obtained through the following method:
i) Construction of the multivariate Cox regression by combination of said biochemical markers and said clinical parameters; and
ii) Analyzing said multivariate Cox regression to assess the independent discriminative value of biochemical markers and clinical parameters.

By ‘in vitro method for diagnosing the risk (predicting the probability) of developing a BLE’ according to the invention, it means that the global method comprises steps wherein the analysis of the data are managed in vitro (RILA assay) or ex-vivo (multivariate cox regression model obtained with clinical parameters previously evaluated on patients).

In particular, the present invention is related to an in vitro method for diagnosing the risk of developing breast late effects after radiotherapy in a subject comprising the steps of:
a. Determining the values of at least one biochemical marker relevant to assess an increasing risk of radiation toxicity in a subject, in particular selected from MLA, proteins and/or genes of radiosensitivity, from a biologic sample of said subject, preferably a blood sample of said subject;
b. Determining the level of at least two clinical parameters relevant to assess an increasing risk of radiation toxicity in a subject, in particular selected from age, breast volume, adjuvant hormonotherapy, boost, node irradiation, and tobacco smoking;
c. Combining said data through a multivariate Cox function to obtain an end value to determine the risk of developing BLE;
   wherein a multivariate Cox regression from said multivariate Cox function is obtained through the following method:
i) Construction of the multivariate Cox regression by combination of said biochemical markers and said clinical parameters; and
ii) Analyzing said multivariate Cox regression to assess the independent discriminative value of biochemical markers and clinical parameters.

In a preferred embodiment, said at least one biochemical marker consists in RILA, and said at least two clinical parameters are tobacco smoking habits and adjuvant hormonotherapy. Advantageously, said at least one biochemical marker RILA is based on the response of CD4 and/or CD8, preferably CD8 after radiotherapy (RT).

In a particular embodiment, said at least one biochemical marker is used in a combination with proteins and/or genes of radiosensitivity.

In a particular embodiment, said at least one biochemical marker is used in a combination with proteins of radiosensitivity, preferably selected from the group consisting of AK2, HSPA8, ANX1, APEX1 and ID2.

In another particular embodiment, said at least one biochemical marker is used in a combination with genes of radiosensitivity, preferably selected from the group consisting of TGFbeta, SOD2, TNFalpha, XRCC1.

In a particular embodiment, said at least one biochemical marker is used in a combination with at least one protein of radiosensitivity, particularly at least two proteins of radiosensitivity, more particularity at least three proteins of radiosensitivity, more particularly at least four proteins of radiosensitivity, even more particularly five proteins of radiosensitivity selected from the group consisting of AK2, HSPA8, ANX1, APEX1 and ID2.

### DETAILED DESCRIPTION

### Definitions

By “Breast Late Effect (BLE)” it is meant atrophic skin, telangiectasia, induration (fibrosis), necrosis or ulceration in a subject after radiotherapy (RT). ‘Induration’ and ‘fibrosis’ are synonymous terms (clinical versus medical semantics).

By “subject”, it is meant human.

By “biological sample”, it is meant in particular blood sample, preferably whole blood extract containing white cells, whole blood extract containing lymphocytes and whole blood extract containing T lymphocytes.

By “clinical parameters” it is meant any clinical parameter relevant to assess an increasing risk of radiation toxicity in a subject, in particular selected from age, breast volume, adjuvant hormonotherapy, boost (complement dose of irradiation), node irradiation, and tobacco smoking, as further disclosed in table 1. The clinical parameters according to the invention are preferably selected from the group of tobacco smoking habits, adjuvant hormonotherapy and mixtures thereof.

By “tobacco smoking habits” it is meant that tobacco smoking patient or non-smoking patients as defined hereafter. A tobacco smoking patient is defined consistently as daily smoker, intermittent smoker or non-daily smoker (and given value=1 in the multivariate Cox function), while a non-smoking patient is defined as some-day smoker or never daily smoker (and given value=0 in the multivariate Cox function).

The term “daily smoker” defines a subject that is currently smoking on a daily basis.

The term “Intermittent smoker” has been defined as not smoking on a daily basis (DiFranza et al., 2007; Lindstrom, Isacsson, & the Malmo Shoulder-Neck Study Group, 2002) or as smoking on 1-15 days in the previous month (McCarthy, Zhou, & Hser, 2001).

The term “non-daily smoker” defines a subject smoking at least weekly (but not daily) or less often than weekly; smoking at least 100 cigarettes in the lifetime and currently smoking some days; smoking more than 100 cigarettes in the lifetime, currently smoking some days, and smoking on fewer than 30 of the past 30 days; smoking more than 100 cigarettes in the lifetime and smoking some days or 1-2 days in the previous 30 days; or smoking fewer than 100 cigarettes in the lifetime and smoking in the previous 30 days (Gilpin, White, & Pierce, 2005; Hassmiller et al., 2003; Husten, McCarty, Giovino, Chrismon, & Zhu, 1998; Leatherdale, Ahmed, Lovato, Manske, & Jolin, 2007; McDermott et al., 2007; Tong, Ong, Vittinghoff, & Perez-Stable, 2006; Wortley, Husten, Trosclair, Chrismon, & Pederson, 2003).

The term “some-day smoker” defines a subject having ever smoked 100 cigarettes during the smoker's lifetime and currently smoking on some days (not every day; CDC, 1993; Hassmiller, Warner, Mendez, Levy, & Romano, 2003).

The term “never daily smoker” defines a subject having never smoked daily for 6 months or more (Gilpin et al., 1997).

By “adjuvant hormonotherapy” it is meant a treatment given after surgery, chemotherapy, and/or radiation therapy to lower the chance recurrence of the cancer. Hormone receptor-positive breast cancer depends on hormones called estrogen and/or progesterone to grow. Adjuvant hormonal therapy allows to lower the levels of these hormones in the body or to block the hormones from getting to any remaining cancer cells. Hormonal therapy for hormone receptor-positive breast cancer are selected from tamoxifen, aromatase inhibitors (AIs), such as anastrozole (Arimidex), exemestane (Aromasin), and letrozole (Femara), and ovarian suppression by surgery or by drugs selected from gonadotropin, luteinizing, goserelin (Zoladex) and leuprolide (Lupron).

By “biochemical markers” it is meant any biochemical marker relevant to assess an increasing risk of radiation toxicity in a subject. The biochemical markers according to the invention are selected from the group of RILA biochemical marker (radiation-induced CD4 and/or CD8, preferably CD8 T-lymphocyte apoptosis), proteins of radiosensitivity such as AK2, HSPA8, IDH2, ANX1, APEX1 and genes of radiosensitivity.

As it is well known from man skilled in the art in this technical domain (Ozsahin et al., 1997 and 2005), RILA is the difference of percentage (%) between the rate of radiation-induced lymphocytes apoptosis after an irradiation of biological sample, preferably a blood sample, with 8 Gy X-Rays and the rate of lymphocytes apoptosis without any irradiation (0 Gy). By lymphocytes, it encompasses CD4 and/or CD8 T-lymphocytes, in particular CD8 T-lymphocytes. RILA is also defined as radiation-induced CD8 T-lymphocyte apoptosis (ref Azria et al., 2015).

So by “biochemical marker consisting in RILA”, also named “RILA biochemical marker” according to the invention, it is understood the difference of percentage (%) between the rate of radiation-induced lymphocytes apoptosis after an irradiation of biological sample, preferably a blood sample, with 8 Gy X-Rays and the rate of lymphocytes apoptosis without any irradiation (0 Gy).

As further disclosed in the examples, the rate of lymphocytes apoptosis with and without any radiation is measured according to RILA assay which is commonly known by the man skilled in the art (Ozsahin et al., 1997 and 2005).

By “proteins of radiosensitivity”, it is meant proteins selected in the group consisting of: adenylate kinase (AK2), Heat shock cognate protein 71 kDa (HSC70 or HSPA8), mitochondrial isocitrate dehydrogenase 2 (IDH2), Anexin 1 (ANX1), and DNA-(apurinic or apyrimidinic site) lyase (APEX1), a specific fragment thereof, a nucleic acid encoding the same, and a combination thereof. Such proteins and fragments thereof are disclosed in WO2014/154854. The presence or level of said protein of radiosensitivity is determined by at least one method selected in the group consisting of: a method based on immune-detection, a method based on western blot, a method based on mass spectrometry, a method based on chromatography, or a method based on flow cytometry, and a method for specific nucleic acid detection. These methods are well known by a person skilled in the art of detecting and quantifying compounds, and particularly proteins, wherein the presence and level of expression of proteins can be determined directly or be analyzed at the nucleic level by detecting, and preferably quantifying, protein-specific nucleic acids, and particularly mRNA. In a first step, proteins and/or nucleic acids are isolated from the biological sample. A method according to the invention may include protein extraction, purification and characterization, using well known biochemistry methods.

By “genes of radiosensitivity”, it is meant single nucleotide polymorphisms (SNPs) identified as genes involved in the fibrosis pathway and ROS management. Such of them have been identified individually as candidate genes: TGFbeta, SOD2, TNFalpha, XRCC1. These genes have been sorted out by a genomic assay as disclosed in Azria et al., 2008. The presence or level of said gene of radiosensitivity is determined by usual method known from man skilled in the art, in particular method for detecting and quantifying specific nucleic acid such as PCR and quantitative PCR. In a particular embodiment, the method as disclosed in Azria et al., 2008 includes lymphocyte isolation, DNA extraction and amplification, and denaturating high-performance liquid chromatography or the Surveyor nuclease assay using a Transgenomic WAVE High Sensitivity Nuclei Acid Fragment Analysis System. PCR primers for the DNA amplicons encompassing the SNPs of interest disclosed above were designed using the genomic sequence obtained from the NCBI.

By “radiotherapy” it is referred to a treatment involving the use of high-energy radiation such as X-rays, gamma rays, electron beams or protons, to kill or damage cancer cells and stop them from growing and multiplying.

RILA Assay

Such RILA assay commonly comprises the following steps:
a) cell culture of blood samples
b) sample irradiation at 8 Gy using a linear accelerator, and
c) sample labelling for evaluation of T-lymphocytes apoptosis, in particular with FACS analysis.

The man skilled in the art may use classical methods to manage the RILA assay.

The evaluation of T-lymphocytes apoptosis at the step c) may use alternative methods such as dosage of Anexin 5, dosage of caspases or FACS analysis, preferably FACS analysis.

Some details for each step a) to c) are disclosed hereunder as particular embodiments.

Cell culture of blood samples:
Before radiotherapy (RT) one blood sample was collected from each patient in heparinized tubes and a volume of blood was aliquoted into well plate;
The Assay is generally carried out in triplicate (0 Gy and 8 Gy).
A culture medium is added (ex: add 2 ml of medium RPMI1640 (Gibco), supplemented with 20% FCS) per well in 6 well tissue culture plates.
Place the tissue culture plates in culture for 24 hours at 37° C., 5% CO2.

Sample irradiation at 8 Gy using a linear accelerator:
After 15 to 30 hours of culture, in particular after 24 h, the plates are safely transported to the irradiator and handle to a radiation technologist, for applying an irradiation of 8 Gy with a dose rate of 1 Gy/min.
In a particular embodiment, the conditions of the irradiation are:
   Dose rate=1 Gy/min
   Energy=6 MV
   Depth=15 mm polystyrene on cells.
   DSP (Distance between Source and Plate)=145 cm
   Field=25*25 cm² collimator.
   Linear accelerator (machine output: 200 UM/mn): 200 UM for 1 Gy: 1600 UM for 8 Gy.
Control cells were removed from the incubator and placed for the same period of time but without radiation treatment.
Incubate the plates for 40 to 50 hours at 37° C., 5% CO2.

Sample labelling for evaluation of T-lymphocytes apoptosis, in particular with FACS analysis:
after 40 to 50 hours incubation, in particular 48 h of incubation, the plates are removed from the incubator and placed at room temperature;
the content of each well is transferred to pre-labeled centrifuges tubes, and after centrifugation (ex: 5 mn at 1450 rpm), the content is labeled with anti-human CD4 and/or CD8-FITC antibody, preferably CD8-FITC antibody;
after addition of lysis buffer, reagents were added to each tube for evaluating lymphocytes apoptosis according to usual methods known from the man skilled in the art, in particular according to flow cytometry method (FACS) with propidium iodide and RNase A as reagents.

Multivariate Cox Regression

Multivariate Cox regression is one of the usual statistical model for time-to-event analysis (Cox, et al. 1984). Apart from a classification algorithm which directly deals with binary or multi-class outcomes, multivariate Cox regression defines a semi-parametric model to directly relate the predictive variables with the real outcome, which is in general a survival time (e.g., in months or years). Multivariate Cox function is the best hazard function in terms of discrimination for time-to event endpoint to combine independent parameters. According to the present invention said independent parameters are biochemical markers and/or clinical parameters related to the development of BLE in a subject, e.g. RILA, adjuvant hormonotherapy, and tobacco smoking habits.

The multivariate Cox function is obtained by combining the relative weight of each parameter, as individually determined in the multivariate Cox regression, with a negative sign when the markers harbor a negative correlation with the observation of fibrosis.

In the present invention, the classification of the patients was made based on the detection of BLE during the clinical follow-up of studies.

Modelling is based on a multicentre population of breast cancer patients treated by radiotherapy and conserving surgery (also named ‘reference population’). The steps to build up the model consisted in:
the identification of biomarkers (e.g, RILA and clinical parameters relevant to assess an increasing risk of radiation toxicity in a subject);
the use of said identified biomarkers in a multicentre clinical trial to identify relevant covariables as prognostic factors of BLE;
the application of these variables on the large multicentre clinical trial to identify the predictive role of combined biochemical marker (RILA) and clinical parameters of BLE.

By “multicenter research trial” it is meant a clinical trial conducted at more than one medical center or clinic.

According to the present invention, the multivariate Cox function is:

Hazard (experiencing a breast late fibrosis)=baseline hazard*exp((β1*biochemical marker)+β2*(Clinical parameter 1)+β3*(Clinical parameter 2)+ . . . βn*(Clinical parameter (n−1) with n superior or equal to 3), where the baseline hazard corresponds to the hazard of experiencing the event (BLE) when all covariates are zero.

The right-hand side of the above equation specify the underlying function of the model. The left-hand side of the equation is the predicted probability that is presented in a nomogram and communicated to the patient. Beta coefficients must be estimated for each covariate and converted to hazard ratios as a measure of effect, as in any statistical report. To obtain the predicted probability of the event in question (experiencing the breast late fibrosis), the above equation is calculated using a patient's individual characteristics and the model-derived beta coefficients.

The baseline hazard is a constant corresponding to the basal risk to develop a BLE without any co-variables. The modelling according to Cox regression model gives this baseline hazard from data coming from a reference population as disclosed above.

Clinical parameters ‘1’ to ‘n’ are selected in the group consisting of: age, breast volume, adjuvant hormonotherapy, boost (complement dose of irradiation), node irradiation, and tobacco smoking, as further disclosed in table 1.

For clinical parameter ‘age’, the median age is 55 years to define patients being 55 years old or less (and given value=0 in the multivariate Cox function) and patients being older than 55 years old (and given value=1 in the multivariate Cox function).

For clinical parameter ‘tobacco smoking’, a tobacco smoking patient is defined consistently as daily smoker, intermittent smoker or non-daily smoker (and given value=1 in the multivariate Cox function), while a non-smoking patient is defined as some-day smoker or never daily smoker (and given value=0 in the multivariate Cox function).

In a particular embodiment, Hazard (experiencing a breast late fibrosis)=baseline hazard*exp((β1*biochemical marker)+β2*(Clinical parameter 1 [0=no; 1=yes])+β3*(Clinical parameter 2 [0=no; 1=yes])), where the baseline hazard corresponds to the hazard of experiencing the event (BLE) when all covariates are zero, and preferably Hazard (experiencing the breast late fibrosis)=baseline hazard*exp(β1*RILA biochemical marker+β2*(Tobacco smoking [0=no; 1=yes])+β3*(Adjuvant hormonotherapy [0=no; 1=yes]), where the baseline hazard corresponds to the hazard of experiencing the event (BLE) when all covariates are zero.

Hazard (experiencing a breast late fibrosis) is also named instantaneous risk to develop BLE for a subject in the description.

Based on this multivariate Cox function, the skilled person would be able to introduce any additional relevant biochemical marker(s) and/or clinical parameter(s) to said multivariate Cox function.

The choice of the optimal model of the invention is assessed by the Harrell's C-index for censored observations and is equal to the probability of concordance between two survival distributions (Harrell and Shih 2001). The C-index or concordance index quantifies the level of concordance between predicted probabilities and the actual chance of having the event of interest.

In one embodiment, the prognosis method of the invention has a Harrell's C-index of 0.6876.

The different coefficients used for the values obtained for the different markers in the multivariate Cox regression can be calculated through statistical analysis, as described in the examples.

In a preferred embodiment, the said multivariate Cox function according to the invention consists of:
Multivariate Cox function (experiencing the BLE)=baseline hazard*exp(β1*RILA+β2*(Adjuvant Hormonotherapy [0=no; 1=yes])+β3*(Tobacco smoking habits[0=no; 1=yes]), wherein:
   β1 is comprised between −0.077 and −0.010;
   β2 is comprised between 0.283 and 1.980;
   β3 is comprised between −0.063 and 0.965.

By ‘Multivariate Cox function (experiencing the BLE)’ in the formula above, it means Hazard according to Multivariate Cox function (experiencing the BLE), also named Hazard Ratio according to Multivariate Cox function (experiencing the BLE); this model comparing 2 populations of patients.

End-Value for a Patient and Uses Thereof

The instantaneous risk to develop a BLE or ‘end-value’ for each patient is estimated taken into account the basal risk (baseline characteristics) and co-variables (clinical parameters);

The ‘end value’ is the predicted probability of occurrence of an event for each patient.

With these combined parameters, a nomogram is a popular visual plot to display the predict probabilities of occurrence of an event for decision support.

To build this nomogram after fitting the cox multivariate model, a linear predictor is obtained according to the method described by Iasonos et al. (2008).

In a particular embodiment, the process for determining the probability of developing a BLE (‘end-value’) of each patient comprises:
1. Description of RILA and clinical parameters relevant to assess an increasing risk of radiation toxicity in a subject, in particular selected from age, breast volume, adjuvant hormonotherapy, boost (complement dose of irradiation), node irradiation, and tobacco smoking, as further disclosed in table 1;
2. Univariate analysis (estimation for each parameter one by one in order to select all significant parameters with p-value<=0.2) under Cox regression model;
3. Multivariate analysis (estimation including all selected parameters by univariate analysis+adding optional non-significant parameters which are clinically relevant) under Cox regression model;
4. Selection of significant parameters and/or clinically relevant to obtain the final model whose linear predictor were extracted to estimate the risk (probability) to develop a BLE; linear predictor were integrated in the software;
5. Execution of software to build a nomogram according to Iasonos et al. 2008 (linear predictor between 0-100 for each parameter including main effect, interaction and piecewise linear effect). This representation gives the risk (probability) of developing a BLE by calculation of an end-value after radiotherapy for each patient according to each individual parameter.

Depending on the end value obtained with the process of statistical analyses (multivariate Cox function and extraction of linear predictor) according to the invention and by the analysis with the multivariate Cox regression, it is possible to predict for a patient the risk of developing a BLE during follow-up after radiotherapy. For example, ‘end value’ of 92% means a 8% risk to developing BLE.

Determining the end-value for a patient, will help the physician to adapt the dose and sequences of radiotherapy treatment to the patient to limit the breast late effects.

In a particular embodiment, wherein the end value of the multivariate Cox function of the method according to the invention is used for the choice of a suitable treatment for the patient, such as an appropriate radiotherapy regimen, or to choose between a mastectomy or conserving surgery, preferably if said end value is more than 20% (cut-off value defined by experts) a decision of mastectomy instead of conserving surgery would be considered, and conversely. The volume of irradiation and the prescription dose will be discussed according to the level of risk i.e. when said end value is more than 8% (cut-off value obtained from the nomogram including all independent predictive factors) there is a risk of developing a BLE after radiotherapy. Absence of boost radiotherapy, absence of node irradiation and dose per fraction less than 2.5 Gy will be different treatment possibilities in case of high risk of BLE and low risk of recurrences of optimal clinical benefit.

In a particular embodiment, the end value of the said multivariate Cox function is used for the choice of a suitable treatment for the patient, such as an appropriate radiotherapy dosage regimen, wherein:
if the patient presents a risk to develop breast late effect, the appropriate radiotherapy dosage regimen will be decreased for example by delivery of partial-breast hypofractionated treatment;
if the patient presents low risk or no risk to develop breast late effect, the appropriate radiotherapy dosage regimen will be increased, for example by delivery of hypofractionated treatment (5 or 16 fractions, which are the common numbers of fractions in such treatment). In particular, if the risk to develop a BLE is more than 8%, we consider it is a (high) risk patient.

In particular, if the risk to develop a BLE is less than 8%, so we consider it is a low risk patient.

In another embodiment, the end value of the multivariate Cox regression of the method according to the invention is used in the decision of performing an immediate breast reconstruction after conserving surgery or mastectomy, preferably if said end value is less than 8% said immediate breast reconstruction after conserving surgery or mastectomy would be considered.

Nomogram

Another object of the present invention is directed to user friendly interface, i.e. nomogram, computer or calculator, implementing said multivariate Cox function, to help physician to interpret the risk of developing BLE after RT. Accordingly, the present invention encompasses a nomogram implementing the said multivariate Cox function according to the invention.

As used herein, “a nomogram” refers to a graphical representation of prognosis formula(ae) from multivariate Cox modelling which allows for estimation of the risk of developing of BLE in a subject, e.g., based on one or more readily obtained parameters, including, but not limited to, RILA, adjuvant hormonotherapy, tobacco smoking habits and proteins of radiosensitivity such as AK2, HSPA8, IDH2, ANX1, APEX1 and/or genes of radiosensitivity. The usefulness of a nomogram is that it maps the predicted probabilities into points on a scale from 0 to 100 in a user-friendly graphical interface. The total points accumulated by the various covariates correspond to the predicted probability for a patient.

According to a preferred embodiment, the steps b) and c) of the method according to the invention can be performed by implementing the data obtained in step a) to a computer or a calculator that will calculate the multivariate Cox regression and the risk of developing of BLE. The data obtained by the physician is therefore more easily interpretable, and will allow for an improvement in the process for deciding the need of performing an immediate breast reconstruction after conserving surgery or mastectomy.

Kits

Another object of the present invention is related to a kit for collecting data of a subject to be further used for detecting the risk of developing of BLE in said subject comprising:
a box/container and bag suited for biological transportation of biological sample, in particular blood sample and
forms to be completed by the patient and/or the nurse and/or the physician, specifically designed and necessary to run the radiosensitivity test and the nomogram analysis.

As example, the forms may contain specific questions aimed at collecting information necessary to run the predictive analysis such as whether the patient has undergone or will undergo adjuvant treatment (chemotherapy, hormone therapy), tobacco habit and date and time when the blood sample was taken.

Another object of the present invention is related to a kit for detecting the risk of developing of BLE in a subject comprising:
reagents for determining the values of at least one biochemical markers according to the invention,
optionally means of collecting information on at least two clinical parameters according to the invention, such as a survey, and
optionally a nomogram according to the invention.

By ‘reagents’ for determining the values of at least one biochemical marker according to the invention, it means in a particular embodiment some or all specific reagents required to run the RILA assay in an independent laboratory, where the irradiation of the sample will be run by a linear accelerator or a lab irradiator

By ‘means of collecting information’ on at least two clinical parameters according to the invention, it means in a particular embodiment specific forms to be completed by the patient and/or the nurse and/or the physician, specifically designed and required to run the radiosensitivity test and the nomogram analysis. In a preferred embodiment, these forms may contain specific questions aimed at collecting information necessary to run the predictive analysis such as whether the patient has undergone or will undergo adjuvant treatment (chemotherapy, hormone therapy), and tobacco habit.

In a particular embodiment, the kit for detecting the risk of developing of BLE in a subject comprises:
some or all specific reagents required to run the RILA assay in an independent laboratory, where the irradiation of the sample will be run by a linear accelerator or a lab irradiator, and
optionally specific forms to be completed by the patient and/or the nurse and/or the physician, specifically designed and required to run the radiosensitivity test and the nomogram analysis.

As example, specific reagents required to run the RILA assay according to the invention may contain PBS, anti-human CD8-FITC and propidium iodide.

Advantageously, the present invention is related to a kit for detecting the risk of developing of BLE in a subject comprising reagents for determining the values of the concentration of RILA, a survey on tobacco smoking habits and adjuvant hormonotherapy consumption, and a nomogram according to according to the invention.

Optionally, the kits according to the invention further comprise a notice of use of said kit.

Computer System

Another object of the present invention is directed to a system including a machine-readable memory, such as a computer or/and a calculator, and a processor configured to compute said multivariate Cox function according to the invention. This system is dedicated to perform the method according to the invention of diagnosis the risk of developing breast late effects (BLE) after radiotherapy in a subject.

In particular embodiment, the said system comprises additionally a module for executing a software to build a nomogram (linear predictor between 0-100 for each parameter including main effect, interaction and piecewise linear effect) and calculate the instantaneous risk (‘end-value’) for the subject to develop a BLE after radiotherapy.

### DESCRIPTION OF THE FIGURES

FIG. 1: ROC Curve for pooled data. The ROC curve was drawn by plotting the sensitivity versus 1-specificity after classification of patients and according to the values obtained for the logistic function for different thresholds (from 0 to 1).

FIG. 2: Scatter plot of RILA according to breast late effect status for pooled data.

### EXAMPLES

Materials and Methods

Patient Analysis

Analysis (pooled data) was performed among 502 breast cancer patients included in the multicentre PHRC (‘Programme Hospitalier de Recherche Clinique’) study evaluating the predictive RILA test in patients treated by radiotherapy and conserving surgery in terms of BLE during the follow-up. 434 patients were eligible for this study. Patients were included between 15 Jan. 2007 and 11 Jul. 2011 and followed with a median follow-up of 38.6 months. A specific questionnaire (Case Report Form) was filled out for each patient including general social demographics administrative data, risk factors, and at each visit, clinical and biological and treatment items, and histologic data described in Table 1. At baseline, 16 patients were excluded for major deviation, one patient withdrew consent before any treatment and blood samples were not technically available for 29 participants. All these patients (n=46) were treated according to current guidelines and were not included for analysis since no data were collected. Thus, RILA and complete RT were performed for 456 patients (90.8%) before entering follow-up. 434 (86.5%) patients were followed for at least 36 months according to the protocol. The 22 other patients interrupted the planned follow-up before 36 months.

Multivariate model was built for a total of 415 patients (‘reference population’) with completed data for the selected parameters.

Radiation-Induced CD8 T-Lymphocyte Apoptosis (RILA) Procedure

The protocol was adapted from studies of Ozsahin et al. (Ozsahin, Crompton et al. 2005). Before RT one blood sample was collected from each patient in a 5-ml heparinized tube. 200 μL of blood was aliquoted into a 6-well plate. All tests were carried out in triplicate for both 0 and 8 Gy. Irradiations (single dose of 8 Gy in a 25 cm×25 cm field size at a dose rate of 1 Gy/min) were delivered after 24 h (H24) using a linear accelerator (2100 EX, 200 UM/min, Varian, US) in the Radiation Department. Control cells were removed from the incubator and placed for the same period of time under the Linac but without radiation treatment. After irradiation, the flasks were immediately incubated at 37° C. (5% CO₂). After a further forty-eight hours (H72), it was labeled with anti-human CD8-FITC antibody (10 μL/tests, Becton Dickinson, USA). After addition of lysis buffer (Becton Dickinson, USA), propidium iodide (Sigma, France) and RNAse (Qiagen, France) was added to each tube and prepared for flow cytometry (FACS).

Preparation and Delivery of Radiotherapy

RT was delivered in the supine position to ensure reproducibility during simulation and treatment. The planning target volume included the whole breast (WB) and the regional lymph nodes (RLN) if necessary. Only photons were allowed for WB irradiation thus allowing standardization of treatment across centers.

A median dose of 50 Gy to the target volume was recommended. The field arrangement involved the use of an anterior photon field in the supraclavicular region and a combination of anterior electrons/photons to the internal mammary nodes at 44-50 Gy. A daily dose of 50 Gy to the WB was delivered by two opposed tangential fields; a boost in the surgical bed up to 10-16 Gy was given when necessary. Fractionation was 2 Gy per fraction, 5 days a week. Calculation used 3-D dosimetry. The ICRU report 62 prescription points were used for prescribing dose. As a minimum, on-line portal imaging was obtained each day for the first three days and once a week during the rest of the course of treatment.

Adjuvant Systemic Therapies

Chemotherapy (CT) regimen when indicated (in case of node positivity and grade 3) consisted either of 6 cycles of FEC 100 [5 FU (500 mg/m 2), epirubicin (100 mg/m 2), cyclophosphamide (500 mg/m 2)] on day 1 and repeated every 21 days or 3 cycles of FEC 100 followed by 3 cycles of docetaxel (100 mg/m 2) every three weeks. In case of HER2 overexpression or gene amplification, trastuzumab (beginning with a loading dose of 8 mg/kg) was added to the protocol (6 mg/kg every 3 weeks for 1 year). Hormonotherapy (HT: tamoxifen or aromatase inhibitor) was started after surgery or after the end of RT and given daily for five years.

End-Point Assessments: Identification of Biomarkers and Relevant Covariables as Prognostic Factors of BLE on Reference Population

The primary objective was the predictive role of RILA in radiation-induced grade BLE (defined as atrophic skin, telangiectasia, induration (fibrosis), necrosis or ulceration). Secondary objectives were the incidence of acute side effects, local recurrence, relapse-free survival (RFS), breast fibrosis-free survival (BF-FS), breast fibrosis-relapse-free survival (BF-RFS) and overall survival (OS). Acute and late side effects were assessed and graded according to the CTC v3.0 scale (Trotti, Colevas et al. 2003).

Toxicity evaluations were performed at baseline, every week during RT, one, three and six months after the last RT fraction, every 6 months up to month 36. Each evaluation was assessed by the physicians blinded for RILA. The most severe BLE observed during the follow-up after RT was considered as the primary endpoint. The most severe late effects (lung, cardiac) observed from 12 weeks to 3 years post RT and the most severe acute side effects (skin and lung mainly) observed from the start of RT to 12 weeks post RT were considered as the secondary endpoints. Toxicities were evaluated using all the possible definitions described in the scale “Dermatology/skin area”, “pulmonary/upper respiratory” and “cardiac general” (Trotti, Colevas et al. 2003).

All endpoints were defined as the interval between the start of RT and following the first events: death for OS, local or contralateral or distant recurrence or death for RFS, grade BLE for BF-FS, and first event of RFS and BF-FS for BF-RFS (Peto, Pike et al. 1977). Censoring patients were patients alive at the last follow-up visit for OS, patients alive and without relapse for RFS, patients alive who never experienced a grade ≥2 BLE for BF-FS and patients alive who never experienced grade BLE or relapse for BF-RFS.

Sample Size Calculation and Statistical Analysis

To test the prognostic value of RILA rate on the occurrence of BLE, we started from the results of our preliminary study (Azria, Gourgou et al. 2004). Details are presented in the following protocol. Briefly, based on a2=0.54, an estimated complication rate of ψ 15% with a two-sided α error of 0.05 and a β error of 0.05 (power=0.95), 430 patients had to be included. The number of patients was increased by at least 15% (n=494) to take into account loss to follow-up and the impact of the boost on BLE.

σ2 is the variance of the studied variable (log CD8) and ψ is the rate of complication/toxicity expected events.

The cumulative incidences of complications as a function of the prognostic variables were calculated using a non-parametric model (Pepe and Mod 1993). The main statistical procedure included a multivariate analysis using the Fine et al. model of competing risks (Fine 2001) for the assessment of the impact of RILA rate on the occurrence of BLE in the presence of other events (such as relapse or death) that are considered as competing risk events in this pathology. For multivariate analysis, selected factors were the baseline parameters with a p-value (statistical significance) less than 0.20 in univariate analysis. Final model was defined using backward stepwise selection (p<0.15) and a step by step method was used to include only the significant parameters (p<0.05) or clinically relevant and/or (p<0.10).

Data were summarized by frequency and percentage for categorical variables and by median and range for continuous variables. Absolute changes in RILA counts before and after irradiation were evaluated as continuous and categorical variables. Three categories were constructed around the 33% quantiles (<12, 12-20, and ≥20) and then merged in two categories (<12 and ≥12).

OS, RFS, BF-FS and BF-RFS rates were estimated by the Kaplan-Meier method (Kaplan and Meier 1958). Ninety-five percent confidence intervals (95% C1) were also determined.

Univariate analysis and multivariate analysis were performed using the Cox proportional hazard's regression model (Cox, et al. 1984) to estimate the hazard ratio including baseline characteristics and treatment parameters. Comparisons were performed using the log-rank test for univariate analysis. Independent effects were evaluated from the likelihood ratio statistics.

Impact of RILA on breast fibrosis-relapse-free survival (BF-RFS) was assessed. The cumulative incidence of BLE and relapse or death were estimated from a competing risk model using estimates obtained from the cause-specific hazard functions and the composite RFS and BF-FS distribution (Arriagada, Rutqvist et al. 1992) and compared using Gray's test.

Median follow-up was estimated with the inverse Kaplan-Meier method. A p-value less than was considered as significant. All statistical tests were two-sided. Stata was used for all statistical analyses (version 13.0) and the SAS macro % cif was used for Gray's test.

To complement analysis, receiver-operator characteristic (ROC) curve analyses for RILA were performed to identify patients who experienced at least a grade 2 BLE during the follow-up (Kramar, Faraggi et al. 2001). The empirical areas under the ROC curves (AUC) and the respective 95% CI were used for RILA to determine the sensitivity, specificity, positive predictive value (PPV), and negative predictive value (NPV).

### Example 1: Descriptive Statistical Analysis for Modelling

The analysis was performed on selected data from studies for all patients observing independent variables, as presented in Table 1 below.

TABLE 1
Prognostic factors characteristics of BLE

| | N = 415 |
|---|---|
| | (‘reference population’) |
| Age (Year) | |
| Mean (std) | 56.52 (9.853) |
| Median (range) | 56.00 (29.00:88.00) |
| Baseline RILA | |
| Mean (std) | 15.96 (8.597) |
| Median (range) | 15.39 (0.74:52.82) |
| Breast Volume (cm³) | n = 410 |
| Mean (std) | 7.47 (13.126) |
| Median (range) | 4.99 (0.00:228.15) |
| Age (Year) | |
| ≤55 | (200/415) 48.2% |
| >55 | (215/415) 51.8% |
| Adjuvant Hormonotherapy | |
| No | (99/415) 23.9% |
| Yes | (316/415) 76.1% |
| Boost (complementary | |
| dose of irradiation) | |
| No | (6/415) 1.4% |
| Yes | (409/415) 98.6% |
| Node irradiation | |
| No | (104/415) 25.1% |
| Yes | (311/415) 74.9% |
| Tobacco smoking | |
| Non smoker | (277/415) 66.7% |
| Ex-smoker or Smoker | (138/415) 33.3% |

According to the CTCAE V3.0 (Trotti, Colevas et al. 2003), toxicity was assessed and patients were divided in two groups (with or without BLE).

The major endpoint was the identification of patients with or without BLE during follow-up. The first stage consisted in identification of factors which differed significantly between these groups by unidimensional analysis and using the log rank test.

The second stage consisted in analysis of multivariate Cox proportional hazard model to assess the independent parameters for the diagnosis of BLE and to estimate the effect size defined as Hazard ratio (HR).

Moreover, the overall diagnosis values were estimated by the Receiving Operating Characteristic curves (ROC Curves). The diagnostic value of RILA was assessed by sensitivity (Se), specificity (Sp), positive and negative predictive values (PPV, NPV).

For such diagnosis or prognosis of a responding phenotype, a responding condition or test outcome is considered a positive result, while a non-responding condition or test outcome is considered a negative result. True and false positive results, NPV, PPV, specificity, sensitivity are defined and calculated as follows:

| | | Condition (responding) | |
|---|---|---|---|
| | | Positive | Negative |
| Test outcome | Positive | True Positive (TP) | False positive (FP) |
| (responding) | Negative | False negative (FN) | True negative (TN) |

PPV=TP/(TP+FP)

NPV=TN/(TN+FN)

Specificity=TN/(TN+FP)

Sensitivity=TP/(TP+FN)

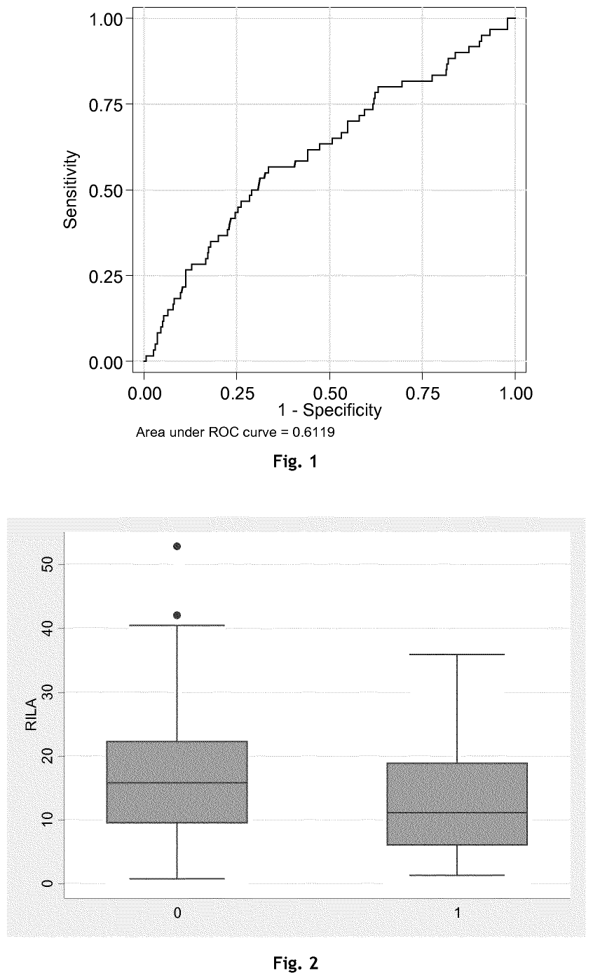

“ROC” or “ROC curve” is a tool for diagnostic test evaluation, wherein the true positive rate (Sensitivity) is plotted in function of the false positive rate (1−Specificity) for different cut-off points of a parameter (FIG. 1) after classification of patients. Each point on the ROC curve represents a sensitivity/specificity pair corresponding to a particular decision threshold (from 0 to 1). The area under the ROC curve (AUC) is a measure of how well a parameter can distinguish between two diagnostic groups (diseased/normal). The accuracy of the test depends on how well the test separates the group being tested into those with and without the disease in question. Accuracy is measured by the area under the ROC curve. An area of 1 represents a perfect test; an area of 0.5 represents a worthless test.

It is usually acknowledged that a ROC area under the curve has a value superior to 0.7 is a good predictive curve for diagnosis. The ROC curve has to be acknowledged as a curve allowing prediction of the diagnosis quality of the method.

The diagnostic value (area under the curve) of RILA marker is presented in table 2.

TABLE 2
Diagnosis value (area under the ROC curve ± sd)
of biochemical markers for significant BLE for the
patients during the follow-up; Sensitivity, specificity
and predictive value of the RILA as a BLE function

| | Pooled data |
|---|---|
| Nb of events (AE) | 60/415 |
| AUC | 0.6119 CI95% [0.531-0.692] |
| cut-off ≥20 | |
| Se | 0.80 |
| Sp | 0.34 |
| PPV | 0.17 |
| NPV** | 0.91 |
| cut-off <12 | |
| Se | 0.55 |
| Sp | 0.67 |
| PPV** | 0.22 |
| NPV | 0.90 |

Se: Sensitivity; Sp: Specificity; PPV: positive predictive value; NPV: negative predictive value.

Results

In the analysis, it has been confirmed that the negative predictive value (RILA with cut-off ≥20) was excellent with more than 90% and may be useful for patients.

In terms of clinical application, patients with high RILA (RILA ≥20) will not observe a BLE and will be proposed to hypofractionation regimen.

Conclusion

The RILA marker is a good marker of BLE during the follow-up leading to develop a personalized treatment according to the patient profile.

The inventors also demonstrated that the combination of RILA marker and two clinical parameters being tobacco smoking habit and adjuvant hormonotherapy, the AUC is improved 0.68 IC95% [0.608-0.749] in comparison to RILA alone (0.61), and for optimal threshold:
Se: 0.80
Sp: 0.487
VPP: 0.209
VPN: 0.935

These results showed that the combination of the three parameters (RILA and two clinical parameters being tobacco smoking habit and adjuvant hormonotherapy), the specificity of the in vitro method is improved and the negative predictive value is even more than 93%.

The following example made with a referenced population of 415 patients, is an illustrative example without limiting the scope of the invention.

### Example 2: Determination of the Multivariate Cox Function

A total of 415 patients with breast cancer and treated by adjuvant radiotherapy after conserving surgery were selected by multivariate Cox regression using independent parameters.

The overall prevalence of BLE was 14.5% (60 events among 415 patients).

Diagnosis of Significant Breast Late Effect (BLE)

Scatter plots were drawn for each study to compare the level of RILA according to BLE status. Patients with BLE presented a low level of RILA.

Moreover, the risk of BLE was higher with low value of RILA. The risk of BLE was significantly increased combined with several clinical parameters (tobacco smoking habits and adjuvant hormonotherapy; Table 3).

Breast late effects (BLE) were evaluated clinically by expert clinicians and graded using the international grading score for toxicity CTCAE V3.0 well known from man skilled in the art (Trotti, Colevas et al. 2003). The NCI Common Terminology Criteria for Adverse Events (CTCAE) v3.0 is a descriptive terminology which can be utilized for Adverse Event (AE) reporting. A grading (severity) scale is provided for each AE term.

TABLE 3
Multivariate analyses to detect independent prognostic factors for Breast Fibrosis
free survival (BF-FS) using the Proportional hazards Cox model (Pooled data).

| Pooled data | N* = 535/703 | Nb of events (AE): 136 | |
|---|---|---|---|
| Median follow-up | HR | CI95% | p-value |
| Univariate model | N = 702 | Nb of events (AE): 60 | |
| RILA | 0.96 | 0.940-0.981 | <0.001 |
| Univariate model | N = 415 | | |
| RILA | 0.96 | 0.927-0.990 | 0.011 |
| Mulivariate model | N* = 415 | Concordance Harrell'C = 0.6876 | |
| RILA | 0.96 | 0.926-0.990 | 0.01 |
| Tobacco Smoking | | | 0.085 |
| No | 1 | | |
| Active/former | 1.57 | 0.939-2.625 | |
| Adjuvant Hormonotherapy | | | 0.009 |
| No | 1 | | |
| Yes | 3.10 | 1.327-7.243 | |
| Mulivariate model** | N* = 415 | Concordance Harrell'C = 0.7004 | |
| RILA | 0.96 | 0.927-0.992 | 0.015 |
| Tobacco Smoking | | | 0.080 |
| No | 1 | | |
| Active/former | 1.59 | 0.946-2.671 | |
| Adjuvant Hormonotherapy | | | 0.015 |
| No | 1 | | |
| Yes | 2.88 | 1.229-6.7573 | |

HR = Hazard ratio estimated by multivariate Cox regression.
RILA = radiation-induced CD8 T-lymphocyte apoptosis
*Number of patients included in the model/included population
**adjusted multivariate model on age (55), Boost(N/Y), node irradiation(N/Y)

Results

Multivariate models identified three parameters (RILA, tobacco smoking and adjuvant hormonotherapy) as independent parameters with an increased risk of BLE for active/former tobacco smoking patients (HR=1.57 CI95%[0.939-2.625]) and for patients treated by hormonotherapy (HR=3.10 CI95%[1.327-7.243]) and a decrease of risk for elevated level of RILA (HR=0.96 CI95% [0.926-0.990]). The other clinical parameters (age, boost and node irradiation) were integrated in multivariate model for adjustment because of clinically relevance. Finally theses parameters were not selected for definitive model.

Conclusion

The combination of clinical parameters (tobacco smoking habits and adjuvant hormonotherapy) and RILA allowed prediction of the probability to develop a breast late effect and integration of clinical and treatment parameters. All these parameters improve the estimation of the risk evaluated only by RILA.

It has been confirmed that the negative predictive value was excellent (more than 90%) and may be useful for patients. In terms of clinical application, patients with low risk of breast recurrences and high RILA will be proposed hypofractionation regimen or partial breast irradiation. The number of fractions will be reduced and higher dose per fraction will be proposed. This scheme will be delivered safely thanks to the RILA assay.

In addition, patients who desire immediate breast reconstruction and radiotherapy will be offered this strategy only in case of low risk (risk less than about 8%) of breast fibrosis evaluated with a high value of RILA.

### Example 3: Construction of a Nomogram Determining the Probability for a Patient of Developing Breast Fibrosis During the Follow-Up after Radiotherapy

Nomogram was built according to the method described by Iasonos et al. (2008) using, as an illustrative example, the estimated parameters by the multivariate Cox function including selected parameters identified as being relevant according to Example 2:

| | |
|---|---|
| RILA | p-value = 0.01 |
| Tobacco smoking | p-value = 0.085 |
| Adjuvant hormonotherapy | p-value = 0.009. |

The optimal beta-coefficients may be obtained by classical statistical analysis and a nomogram may be easily built by a man skilled in the art based on these coefficients and resulted hazard (experiencing BLE).

For example, a patient with a RILA=10% (determined as disclosed above on a blood sample), non-smoker and treated by adjuvant hormonotherapy, we can calculate the following risk step by step:

1/RILA = 10% => 82 points
2/Non-smoker/Tobacco = 0 => 0 points
3/Treated by Adjuvant Hormonotherapy/Adj_HRM = 1 => 46 points
4/Total points: 82 + 0 + 46 = 128 points
5/Probability of a 3y-BF-FS is between 0.85 and 0.80 corresponding
to a risk of developing a breast late effect between 15 and 20%.

### REFERENCES

Al-Ghazal, S. K., L. Fallowfield, et al. (1999). “Does cosmetic outcome from treatment of primary breast cancer influence psychosocial morbidity?” European journal of surgical oncology: the journal of the European Society of Surgical Oncology and the British Association of Surgical Oncology 25(6): 571-573.
Arriagada, R., L. E. Rutqvist, et al. (1992). “Competing risks determining event-free survival in early breast cancer.” Br J Cancer 66(5): 951-957.
Azria, D., M. Betz, et al. (2012). “Identifying patients at risk for late radiation-induced toxicity.” Critical reviews in oncology/hematology 84 Suppl 1: e35-41.
Azria, D., S. Gourgou, et al. (2004). “Concomitant use of tamoxifen with radiotherapy enhances subcutaneous breast fibrosis in hypersensitive patients.” Br J Cancer 91(7): 1251-1260.
Azria, D., M. Ozsahin, et al. (2008). “Single Nucleotide Polymorphisms, Apoptosis, and the Development of Severe late Adverse Effects After Radiotherapy”. Clin Cancer Res 2008 October; 14(19): 6284-6288.
Azria, D., O. Riou, et al. (2015). “Radiation-induced CD8 T-lymphocyte Apoptosis as a Predictor of Breast Fibrosis After Radiotherapy: Results of the Prospective Multicenter French Trial.” EBioMedicine 2(12): 1965-1973.
Cox, D. R., et al., Eds. (1984). Analysis of Survival Data., New York: Chapman & Hall.
Fine, J. P. (2001). “Regression modeling of competing crude failure probabilities.” Biostatistics 2(1): 85-97.
Harrell, F. E., Jr. and Y. C. Shih (2001). “Using full probability models to compute probabilities of actual interest to decision makers.” Int J Technol Assess Health Care 17(1): 17-26.
Iasonos, A., D. Schrag et al. (2008). “How to build and interpret a Nomogram for Cancer Prognosis”. Journal of Clinical Oncology. Review Article. Volume 26, Number 8, March 2008.
Kaplan, E. L. and P. Meier (1958). “Non-parametric estimation from incomplete observation.” J Am Stat Assoc 53: 457-481.
Kramar, A., D. Faraggi, et al. (2001). “mROC: a computer program for combining tumour markers in predicting disease states.” Comput Methods Programs Biomed 66(2-3): 199-207.
Ozsahin, M., N. E. Crompton, et al. (1997). “Rapid assay of intrinsic radiosensitivity based on apoptosis in human CD4 and CD8 T-lymphocytes”. Int. J. Radiation Oncology Biol. Phys., Vol. 38, N^{o} 2, pp. 429440.
Ozsahin, M., N. E. Crompton, et al. (2005). “CD4 and CD8 T-lymphocyte apoptosis can predict radiation-induced late toxicity: a prospective study in 399 patients.” Clin Cancer Res 11(20): 7426-7433.
Pepe, M. S. and M. Mod (1993). “Kaplan-Meier, marginal or conditional probability curves in summarizing competing risks failure time data?” Stat Med 12(8): 737-751.
Peto, R., M. C. Pike, et al. (1977). “Design and analysis of randomized clinical trials requiring prolonged observation of each patient. II. analysis and examples.” Br J Cancer 35(1): 1-39.
Trotti, A., A. D. Colevas, et al. (2003). “CTCAE v3.0: development of a comprehensive grading system for the adverse effects of cancer treatment.” Semin Radiat Oncol 13(3): 176-181.

1. An in vitro method for diagnosing the risk of developing breast late effects (BLE) after radiotherapy in a subject comprising the steps of:
a. Determining the values of at least one biochemical marker relevant to assess an increasing risk of radiation toxicity in a subject, in particular selected from RILA, proteins and/or genes of radiosensitivity, from a biologic sample of said subject, preferably a blood sample of said subject;
b. Determining the level of at least two clinical parameters relevant to assess an increasing risk of radiation toxicity in a subject, in particular selected from age, breast volume, adjuvant hormonotherapy, boost, node irradiation, and tobacco smoking;
c. Combining said data through a multivariate Cox function to obtain an end value to determine the risk of developing BLE;
wherein a multivariate Cox regression from said multivariate Cox function is obtained through the following method:
i) Construction of the multivariate Cox regression by combination of said biochemical markers and said clinical parameters; and
ii) Analyzing said multivariate Cox regression to assess the independent discriminative value of biochemical markers and clinical parameters.

2. The method according to claim 1, wherein said at least one biochemical marker consists in RILA which is based on the response of CD4 and/or CD8 after radiotherapy (RT), preferably based on the response of CD8 after radiotherapy (RT).

3. The method according to claim 1, wherein said at least two clinical parameters are at least tobacco smoking habits and adjuvant hormonotherapy.

4. The method according to claim 1, wherein said biochemical marker is used in combination with proteins and/or genes of radiosensitivity.

5. The method according to claim 4, wherein said proteins of radiosensitivity are selected from the group consisting of AK2, HSPA8, ANX1, APEX1 and ID2.

6. The method according to claim 4, wherein said genes of radiosensitivity are selected from the group consisting of TGFbeta, SOD2, TNFalpha and XRCC1.

7. The method according to claim 1, wherein said multivariate Cox function consist of:
Hazard (experiencing the BLE)=baseline hazard*exp(β1*RILA+β2*(Adjuvant Hormonotherapy [0=no; 1=yes])+β3*(Tobacco smoking habits [0=no; 1=yes]), wherein:
β1 is comprised between −0.077 and −0.010;
β2 is comprised between 0.283 and 1.980;
β3 is comprised between −0.063 and 0.965.

8. The method according to claim 1, wherein the end value of the said multivariate Cox function is used for the choice of a suitable treatment for the patient, such as an appropriate radiotherapy dosage regimen, wherein:
if the patient presents a risk to develop breast late effect, the appropriate radiotherapy dosage regimen will be decreased for example by delivery of partial-breast hypofractionated treatment;
if the patient presents low risk or no risk to develop breast late effect, the appropriate radiotherapy dosage regimen will be increased, for example by delivery of hypofractionated treatment (5 or 16 fractions).

9. The method according to claim 1, wherein the end value of the said multivariate Cox function is used to choose between a mastectomy or conserving surgery, preferably if said end value is more than 20% a decision of mastectomy instead of conserving surgery would be considered, and conversely.

10. The method according to claim 1, wherein the end value of the multivariate Cox regression is used in the decision of performing an immediate breast reconstruction after conserving surgery or mastectomy, preferably if said end value is less than 8% said immediate breast reconstruction after conserving surgery or mastectomy would be considered.

11. The method according to claim 1, wherein the functional representation is a nomogram.

12. A kit suitable for collecting data of a subject comprising:
a box/container and bag suited for biological transportation of biological sample, in particular blood sample and
forms to be completed by the patient and/or the nurse and/or the physician, specifically designed and necessary to run the radiosensitivity test and the nomogram analysis.

13. A kit suitable for detecting the risk of developing of breast late effect (BLE) in a subject comprising:
reagents for determining the values of at least one biochemical markers according to claim 1; and
optionally means of collecting information on at least two clinical parameters according to claim 1.

14. A kit according to claim 13, comprising:
some or all specific reagents required to run the RILA assay in an independent laboratory, where the irradiation of the sample will be run by a linear accelerator or a lab irradiator, and
optionally specific forms to be completed by the patient and/or the nurse and/or the physician, specifically designed and required to run the radiosensitivity test and the nomogram analysis.

15. A system including a machine-readable memory, such as a computer or/and a calculator, and a processor configured to compute said multivariate Cox function according to claim 1 and preferably additionally a module for executing a software to build a nomogram and calculate the instantaneous risk for the subject to develop a BLE after radiotherapy.
